(19) United States
(12) Patent Application Publication
(10) Pub. No.: US 2024/0180838 A1
(43) Pub. Date: Jun. 6, 2024

(54) NANOPARTICLE (NP) COMPOSITIONS AND METHODS OF USE THEREOF

(71) Applicant: RUTGERS, THE STATE UNIVERSITY OF NEW JERSEY, New Brunswick, NJ (US)

(72) Inventors: Maricely Ramirez Hernandez, Linden, NJ (US); Tewodros Asefa, Kendall Park, NJ (US); Jeffrey M. Boyd, Highland Park, NJ (US); Javiera Norambuena-Morales, New Brunswick, NJ (US)

(73) Assignee: RUTGERS, THE STATE UNIVERSITY OF NEW JERSEY, New Brunswick, NJ (US)

(21) Appl. No.: 18/528,873

(22) Filed: Dec. 5, 2023

Related U.S. Application Data
(60) Provisional application No. 63/386,024, filed on Dec. 5, 2022.

(51) Int. Cl. A61K 9/16; A61K 31/167; A61K 33/34

(52) U.S. Cl. CPC A61K 9/167; A61K 9/1611; A61K 31/167; A61K 33/34

(57) ABSTRACT

The present disclosure relates to compositions and methods for treating a wound, or location of interest, in a subject by topically administering to the subject a therapeutic composition of the present disclosure. The composition of the disclosure comprises nanoparticle (NP) composites for delivering at least two cargos to the wound. In certain embodiments, the at least two cargos comprise a metal and a small molecule drug.

### CROSS-REFERENCE TO RELATED APPLICATIONS

This application claims priority under 35 U.S.C. § 119(e) to U.S. Provisional Patent Application No. 63/386,024, filed Dec. 5, 2022, which is incorporated herein by reference in its entirety.

### STATEMENT REGARDING FEDERALLY SPONSORED RESEARCH OR DEVELOPMENT

This invention was made with government support under 1R01AI139100-01 awarded by the National Institute of Allergy and Infectious Diseases and NE1748 awarded by U.S. Department of Agriculture Multistate Award. The government has certain rights in the invention.

### BACKGROUND

Due to increased antimicrobial resistance to conventional antibiotics, there is an urgent need for the development of new antimicrobials and materials that can more effectively deliver antimicrobial agents in a controlled manner. According to the Center for Disease Control (CDC) in the U.S. around 2.8 million people are affected by antibiotic-resistant infections, resulting in approximately 35,000 deaths annually. There are several potential approaches to combat this problem, such as development of new classes of antibiotics, changes in public health protocols, and/or development of novel materials that can deliver antimicrobial agents effectively.

There is thus a need in the art for compositions and/or methods for the effective delivery of bactericidal agents. The present disclosure addresses this need.

### BRIEF DESCRIPTION OF THE FIGURES

The drawings illustrate generally, by way of example, but not by way of limitation, various embodiments of the present application.

FIG. 1 provides a schematic illustration of the synthesized Cu(II) ion- and rafoxanide-loaded antimicrobial mesoporous silica nanomaterials and their expected antimicrobial effects.

FIG. 2 provides a schematic depicting the procedure used for the measurement of Cu(II) ions released antimicrobial nanomaterials via spectrophotometric technique by amplifying the optical signal associated with Cu(II) with polyethyleneimine (PEI).

FIGS. 3A-3B provide graphs depicting N₂ gas adsorption-desorption isotherms (FIG. 3A) and pore size and volume size distributions (FIG. 3B) of SBA-15, DAMO/SBA, Cu/DAMO-SBA, RFX/DAMO-SBA, Cu-RFX/DAMO-SBA, and RFX-Cu/DAMO-SBA nanomaterials.

FIGS. 4A-4B depict thermogravimetry analysis curves for exemplary nanomaterials. FIG. 4A: TG curves for antimicrobial-loaded nanomaterials synthesized by loading Cu(II) species first (i.e., Cu/DAMO-SBA), followed by RFX (i.e., Cu-RFX/DAMO-SBA). FIG. 4B: TG curves for antimicrobial-loaded nanomaterials synthesized by loading RFX first (i.e., RFX/DAMO-SBA), followed by RFX (i.e., RFX-Cu/DAMO-SBA). Parent material (i.e., DAMO-SBA) is included in FIGS. 4A-4B for comparison.

FIGS. 5A-5C depict exemplary X-ray photoemission spectroscopy (XPS) spectra for Cu-RFX/DAMO-SBA and RFX/DAMO-SBA materials. FIG. 5A: survey scans for Cu-RFX/DAMO-SBA and RFX/DAMO-SBA materials. FIG. 5B: high resolution scans for Cl 2p. FIG. 5C: high resolution scans for I 3d.

FIG. 6 provides a photograph demonstrating that copper and rafoxanide have synergistic effects against S. aureus. Photograph displays drop plates containing 10-fold serial dilutions (left to right: 10⁻¹ to 10⁻⁷ dilution) of cultures treated for 18 hours with the indicated materials. Samples and control without addition of sample are shown: (a) control (i.e., no material), (b) SBA-15, (c) DAMO/SBA, (d) Cu/DAMO-SBA-15, (e) RFX/DAMO-SBA, (f) Cu-RFX/DAMO-SBA, and (g) RFX-Cu/DAMO-SBA nanomaterials.

FIG. 7 provides representative photographs of drop plates containing 10-fold serial dilutions (left to right: 10⁻¹ to 10⁻⁷ dilution) of treated cultures of wild-type S. aureus, S. aureus mutants, wild-type E. coli, and E. coli mutants. Control and physical mixtures of actives without the SBA-15 nanomaterials are shown: (a) control (i.e., no material); (b) rafoxanide (RFX), (c) copper(II) acetate; and (d) copper(II) acetate+RFX. The amounts of each are based on TGA (for RFX loading estimate) and inductively coupled plasma-optical emission spectroscopy (ICP-OES) for Cu(II) ions in materials. The mutant strains are deficient in removing Cu ions from the cytosol.

FIGS. 8A-8B provide graphs depicting release profiles for Cu(II) ions and/or RFX-containing materials at pH 7 (FIG. 8A) and pH 5 (FIG. 8B).

FIGS. 9A-9C provide bar graphs depicting sustained release profiles of Cu(II) ions from exemplary nanomaterials over 5 cycles at different pH values (i.e., pH 7 and pH 5) measured at the absorption maximum of (max=275 nm) for Cu/DAMO-SBA (FIG. 9A), Cu-RFX/DAMO-SBA (FIG. 9B), and RFX-Cu/DAMO-SBA (FIG. 9C) nanomaterials.

FIG. 10 provides a schematic depicting the synthesis of exemplary Cu(II) and/or rafoxanide loaded nanomaterials, wherein the sequence of synthetic steps is highlighted.

FIG. 11 provides results of dose-dependent experiments using certain exemplary materials of the present disclosure against coagulase-positive (cop) Staphylococcus aureus. Representative photographs display drop plates containing 10-fold serial dilutions (left to right: 10⁻¹ to 10⁻⁷ dilution) of S. aureus mutant strains that cannot efflux copper ions from the cytosol using different materials dosages (i.e., 5 mg, 10 mg, 20 mg, and 40 mg, respectively). Cultures were treated for 18 hours with the indicated material before serial dilution and spot plating on tryptic soy broth (TSB) medium (left to right: 10⁻¹ to 10⁻⁷ dilutions).

FIG. 12 provides a photograph demonstrating that copper and rafoxanide have synergistic effects against S. aureus. Photograph displays drop plates containing 10-fold serial dilutions (left to right: 10⁻¹ to 10⁻⁷ dilution) of cultures treated for 18 hours with the indicated materials. Samples and control without addition of sample are shown: (a) control (i.e., no material), (b) SBA-15, (c) DAMO/SBA, (d) Cu/DAMO-SBA-15, (e) RFX/DAMO-SBA, (f) Cu-RFX/DAMO-SBA, (g) RFX-Cu/DAMO-SBA nanomaterials, (h) rafoxanide, (i) clindamycin (CDM), (j) erythromycin (ERM), and (k) trimethoprim (TRM). Approximately 20 mg of payload used in the preparation of each loaded material, and approximately 20% of the payload in each material is released in the biological media. Minimum inhibitory concentrations (MICs) of CDM, ERM, and TRM, as reported in the literature, are 0.19 μg/mL, 32 μg/mL, and 0.19 μg/mL, respectively.

### BRIEF SUMMARY

In one aspect, the present disclosure provides a nanoparticle (NP) functionalized with at least one pH-responsive moiety. In certain embodiments, in the NP at least two cargos are associated with the NP and/or the at least one pH-responsive moiety. In certain embodiments, each cargo is independently selected from the group consisting of a therapeutic cargo, a molecular marker, and/or a biomarker. In certain embodiments, the at least two cargos comprise a metal and a small molecule drug. In certain embodiments, the metal is Cu(II). In certain embodiments, the small molecule drug is rafoxanide. In certain embodiments, swelling of at least one the pH-responsive moiety allows for release of the at least two cargos associated with the NP and/or at least one pH-responsive moiety.

In another aspect, the present disclosure provides a method of preparing a nanoparticle of the present disclosure. In certain embodiments, the NP is functionalized with at least one pH-responsive moiety. In certain embodiments, the NP comprises at least two cargos. In certain embodiments, the at least two cargos comprise a first cargo and a second cargo. In certain embodiments, NP is prepared by contacting the first cargo comprising a metal with the NP functionalized with at least one pH-responsive moiety to prepare a metal-loaded NP. In certain embodiments, the NP is prepared by contacting the second cargo comprising a small molecule drug.

In another aspect, the present disclosure provides a composition. In certain embodiments, the composition comprises about 0.10% to about 0.25% of chitosan. In certain embodiments, the composition comprises about 0.10% to about 2.0% of sodium alginate. In certain embodiments, the composition comprises about 0.1% to about 0.5% of gelatin. In certain embodiments, the composition comprises about 0.01% to about 1% of grapeseed oil. In certain embodiments, the composition comprises poly-vinyl-alcohol (PVA). In certain embodiments, the composition comprises aloe vera gel. In certain embodiments, the composition comprises at least one NP of the present disclosure. In certain embodiments, the composition is formulated for application to a wound of a subject for promoting healing of the wound.

In another aspect, the disclosure further provides a kit comprising at least one composition of the disclosure, at least one applicator, and instructional material for use thereof. The instructional material included in the kit comprises instructions for carrying out the method of the disclosure.

In one aspect, the disclosure further provides a kit comprising at least one composition of the disclosure, at least one applicator, and instructional material for use thereof. The instructional material included in the kit comprises instructions for carrying out the method of the disclosure.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

Repurposing available, low-cost drugs to fight against bacterial infection represents a strategy to circumvent the challenges of antibiotic resistance while new classes of antibiotics are designed and brought to commercial use. Recently, researchers have investigated the effect of salicylanilide anthelmintic drugs such as rafoxanide, niclosamide, and closantel against gram-positive and gram-negative bacteria.

Staphylococcus aureus is one of the most common species in nosocomial infections and is ubiquitous in the biofilms that are present in wounds. Several studies have thus been aimed at tackling this microbe and its effects on the population. Recent studies have shown the effect of rafoxanide and related compounds against planktonic and biofilm-forming Staphylococcus aureus. Rafoxanide and other anthelmintic compounds and their analogs can serve as GroEL/ES inhibitors, which are molecular chaperones and proteins that promote cell viability and stability. Inhibition of the GroEL/ES system has recently been explored as an alternative mechanism against antibiotic-resistant bacteria since it is essential for bacterial proliferation. As the GroEL/ES chaperonin system is essential for growth under all conditions, developing small molecules that can inhibit its function has particularly been proven a viable antibacterial strategy.

While rafoxanide has primarily been used in livestock to treat liver flukes such as Fasciola hepatica and Fasciola gigantica species, loading rafoxanide within a solid nanostructured system like mesoporous silica may provide an alternative way to deliver and effectively use this drug in the battle against antibiotic-resistant pathogens in humans. Furthermore, combining such small molecule nonantibiotic drugs with metals and metal ions can further increase their bactericidal properties through combinatorial and/or synergistic effects.

One of the most promising alternatives against antimicrobial resistance and bactericidal strategies is to destabilize the cell walls of microbes by using silver and copper ions or nanoparticles. One caveat limiting the use of these metallic species is the propensity of these systems to undergo reduction and aggregate due to their high surface energy. Therefore, capping agents and mesoporous hosts are widely accepted strategies to control the aggregation of metallic agents in form of nanoparticles and the release of the payloads of the bioactive agents held therein. Further, such mesoporous drug delivery vehicles containing active agents could easily be further functionalized and processed in formulations.

Previous studies have demonstrated the synthesis of phosphate-functionalized SBA-15 mesoporous silica nanomaterial containing copper ions for antibacterial applications against Escherichia coli. Further, it has been shown that copper ions incorporated using the phosphate ligands improved the antimicrobial performance of the nanomaterial when compared to copper ions loaded in the same matrix without the ligands. Additionally, it has been shown that even a small amount of copper ions (>10%) may serve as promising antibacterial agents for biomedical applications and to control microorganisms on surfaces.

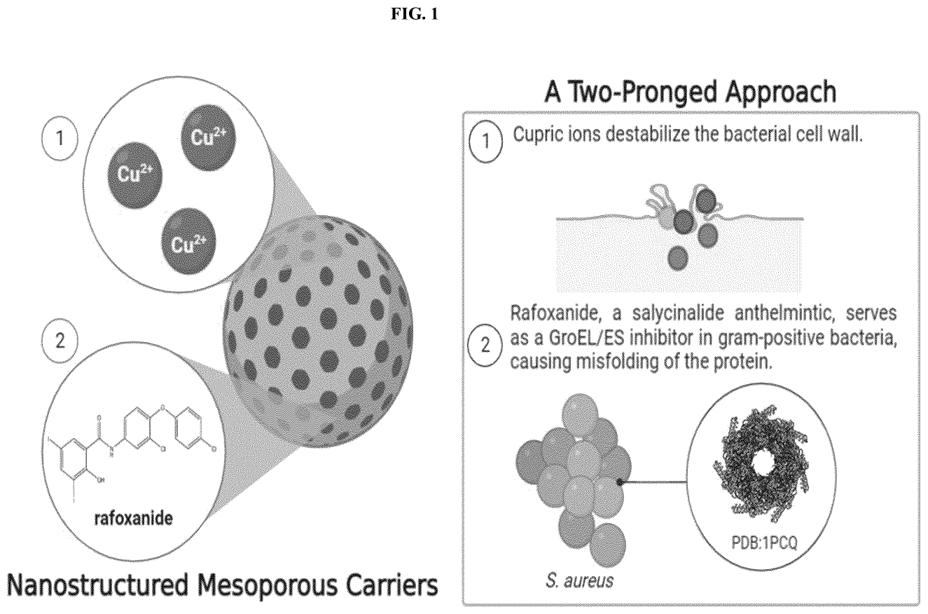

Described herein is the synthesis of amine-functionalized mesoporous silica nanomaterial with, not only anchored Cu(II) ions, but also physisorbed rafoxanide, which shows synergistic antimicrobial efficacy against wild-type and copper-sensitive strains of Staphylococcus aureus and Escherichia coli. The structural characteristics of the synthesized materials, the release of copper ions from the system using spectrophotometric techniques, and their bactericidal performances with respect to relevant control materials, are further described herein. A rendering of the synthesized material containing both components (i.e., Cu(II) ions and rafoxanide) is shown in FIG. 1.

In certain embodiments, the system results in a two-pronged antimicrobial effect, wherein Cu(II) ions are believed to serve as cell wall destabilizers, inhibitors of cytosolic protein function, and promoters of protein aggregation, whereas rafoxanide is believed to function as a GroEL/ES inhibitor, resulting in the accumulation of misfolded of proteins. Furthermore, the mesoporous silica nanomaterials are found to facilitate improved and controlled delivery of both antimicrobial agents (i.e., Cu(II) and rafoxanide).

Four types of SBA-15 type mesoporous silica nanomaterials containing Cu(II) ions, rafoxanide, and both components were synthesized and characterized. Structural and compositional characterizations have shown that both rafoxanide and Cu(II) species are contained within the SBA-15 nanomaterials. The oxidation state of copper remained unchanged (i.e., as Cu(II)) throughout the synthesis of the nanomaterials, as confirmed via XPS spectroscopy. The results showed that, compared with materials where there is only rafoxanide present, SBA-15 mesoporous silica nanocarriers loaded with rafoxanide and copper show ten-fold bactericidal activity against wild-type S. aureus.

Interestingly, the bactericidal effect of the nanomaterial was substantially affected (improved) by the synthetic sequence applied. The nanomaterials where rafoxanide was loaded after Cu(II) ions were immobilized were found to be more effective than if the sequence were reversed (rafoxanide or RFX before Cu(II) species). The present disclosure, in one aspect, demonstrates that combinations of copper and anthelmintic can serve as alternative, potent antimicrobial agents against antibiotic-resistant bacteria while giving insight into how formulation can affect their performances. The present disclosure further demonstrates an alternate carrier for RFX that is lighter and more thermally stable than standard methods for RFX administration (e.g., suspensions or solid dispersions).

The disclosure of PCT International Patent Application No. PCT/US2021/032211, filed May 13, 2021, and U.S. Provisional Application No. 63/025,306, filed May 15, 2020, are incorporated herein by reference in their entireties.

### Definitions

As used herein, each of the following terms has the meaning associated with it in this section.

Unless defined otherwise, all technical and scientific terms used herein generally have the same meaning as commonly understood by one of ordinary skill in the art to which this disclosure belongs. Generally, the nomenclature used herein and the laboratory procedures in organic chemistry, formulation chemistry, and biology are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or +10%, more preferably +5%, even more preferably +1%, and still more preferably +0.1% from the specified value, as such variations are appropriate to perform the disclosed methods.

The term “anthelmintic” as used herein refers to a group of antiparasitic drugs used in the treatment and/or prevention of nematode, fluke, cestode, and/or trematode infections and/or related diseases or disorders. Non-limiting examples of anthelmintics include rafoxanide (i.e., N-[3-Chloro-4-(4-chlorophenoxy)phenyl]-2-hydroxy-3,5-diiodobenzamide), niclosamide, benzimidazoles, avermectins, diethylcarbamazine, pyrantel pamoate, levamisole, salicylanilide, nitazoxanide, oxamniquine, praziquantel, artemisinin, piperazine, and praziquantel, inter alia.

The term “biocompatible” as used herein refers to the ability of a material to perform appropriately when in contact with living tissue without damaging the material or the tissue which are in contact, or any further tissue and/or component of the organism in which the material is direct or indirect contact with.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “dendrimer” or “dendrimeric” as used herein refers to highly ordered, branched polymeric molecules.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “to dress a wound” refers to the act of applying an adjunct to the wound, in order to improve healing and/or prevent further harm.

As used herein, the term “effective” means adequate to accomplish a desired, expected, or intended result.

As used herein, the terms “effective amount,” “pharmaceutically effective amount” and “therapeutically effective amount” refer to a nontoxic but sufficient amount of an agent to provide the desired biological result. That result can be reduction and/or alleviation of the frequency and/or severity of signs, symptoms, or causes of a disease, or any other desired alteration of a biological system. An appropriate therapeutic amount in any individual case may be determined by one of ordinary skill in the art using routine experimentation.

As used herein, the terms “inhibiting,” “reducing,” and variations of these terms, include any measurable decrease, such as but not limited to complete or substantially complete inhibition.

As used herein, the “instructional material” includes a publication, a recording, a diagram, or any other medium of expression that may be used to communicate the usefulness of the compounds of the disclosure. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds of the disclosure or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

As used herein, the term “or” means “and/or,” unless explicitly indicated to refer to alternatives only or the alternatives are mutually exclusive, although the disclosure supports a definition that refers to only alternatives and “and/or.”

As used herein, the terms “patient” and “subject” refer to a human or a non-human animals. Non-human mammals include, for example, livestock and pets, such as ovine, bovine, porcine, canine, feline and murine mammals. Preferably, the subject is human.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the disclosure with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

As used herein, the term “preventing” as relating to a condition in a subject refers to the ability of avoiding the onset of the condition in a patient that is likely, susceptible, or expected to develop the condition.

The term “therapeutic” as used herein means a treatment and/or prophylaxis. A therapeutic effect is obtained by suppression, remission, or eradication of a disease state.

As used herein, the term “topical” as applied to mode of administration includes but is not limited to “dermal.” The term “dermal” refers to the application of a composition to the skin of a subject. The term “topical refers to the application of a composition to the body's natural surface, which has not been created by surgical intervention or any artificial means.

As used herein, the terms “treat,” treating,” “treatment,” and the like, including “healing,” refer to reducing or improving a disease or condition and/or symptom associated therewith. It will be appreciated that, although not precluded, treating a disease or condition does not require that the disease, condition or symptoms associated therewith be completely ameliorated or eliminated.

As used herein, the term “wound dressing” refers to an adjunct (such as a chemical and/or material) used by a person for application to a wound to promote healing and/or prevent further harm.

Throughout this disclosure, various aspects of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5, and 6. This applies regardless of the breadth of the range.

The recitation of an embodiment for a variable or aspect herein includes that embodiment as any single embodiment or in combination with any other embodiments or portions thereof.

### Compositions

### Nanoparticles (NPs)

In one aspect, the present disclosure provides a nanoparticle (NP) functionalized with at least one pH-responsive moiety.

In certain embodiments, in the NP comprises at least two cargos. In certain embodiments, the at least two cargos are independently associated with the NP and/or the at least one pH-responsive moiety. In certain embodiments, each cargo is independently selected from the group consisting of a therapeutic cargo, a molecular marker, and/or a biomarker.

In certain embodiments, swelling of at least one the pH-responsive moiety allows for release of the at least two cargos associated with the NP and/or at least one pH-responsive moiety.

In certain embodiments, the functionalization of the nanoparticle with the pH-responsive moiety comprises a non-covalent (e.g., hydrogen bonding, ionic, or hydrophobic interaction).

In certain embodiments, the functionalization of the nanoparticle with the pH responsive moiety comprises a covalent bond between the nanoparticle with the pH responsive moiety. In certain embodiments, the covalent bond comprises an Si—O bond. In certain embodiments, the Si—O bond forms between an oxygen atom of a silicate (silicon oxide) or titanate (titanium oxide) moiety at the surface (e.g., internal pore surface or nanoparticle surface) of the nanoparticle and a silicon atom of a reagent comprising the pH-responsive moiety. In certain embodiments, the reagent comprising the pH-responsive moiety is a trialkoxysilicate substituted with a pH-responsive moiety. In certain embodiments, the Si—O bond is formed by reaction of a nanoparticle surface siloxide or silanol (i.e., Si—O— or Si—OH) and a trialkoxysilicate (e.g., R′₃OSi—R″), wherein the trialkoxysilicate is substituted with a pH-responsive moiety. In certain embodiments, the trialkoxysilicate substituted with a pH-responsive moiety is N-(2-aminoethyl)-3-aminopropyltrimethoxysilane (DAMO).

In certain embodiments, the pH-responsive non-covalently associates with the at least one cargo. In certain embodiments, the non-covalent association of the pH-responsive moiety and the at least one cargo comprises a hydrogen bonding interaction. In certain embodiments, the non-covalent association of the pH-responsive moiety and the at least one cargo comprises an ionic interaction. In certain embodiments, the non-covalent association of the pH-responsive moiety and the at least one cargo comprises a hydrophobic interaction. In certain embodiments, the non-covalent association of the pH-responsive moiety and the at least one cargo is disrupted by a change in pH. In certain embodiments, disruption of the non-covalent association of the pH-responsive moiety and the at least one cargo facilitates release of the at least one cargo from the LNP.

In certain embodiments, the therapeutic cargo is at least one selected from the group consisting of a small molecule drug, a metal species, and a therapeutic peptide.

In certain embodiments, the therapeutic cargo comprises at least one selected from the group consisting of a cleaning agent, disinfecting agent, preserving agent, healing agent, bacteriostatic agent, antifungal agent, and antiviral agent.

In certain embodiments, the small molecule drug is a GroEL/ES inhibitor. In certain embodiments, the GroEL/ES inhibitor is selected from the group consisting of hydroxybiphenylamide and sulfamido-2-arylbenzoxazole, or an analogue thereof, and any combination thereof.

In certain embodiments, the small molecule drug is a anthelmintic. In certain embodiments, the anthelmintic is rafoxanide.

In certain embodiments, the metal species is Cu(II).

In certain embodiments, the cargo comprises rafoxanide and Cu(II).

In certain embodiments, the NP is prepared by loading the Cu(II) into the NP before loading the rafoxanide into the NP.

In certain embodiments, the cargo is a molecular marker and/or a biomarker. In certain embodiments, the molecular marker and/or biomarker detects for the presence of bacteria. In certain embodiments, the molecular marker and/or biomarker is selected from the group consisting of O₂, β-catenin and c-myc, and matrix metalloproteinases.

In certain embodiments, the at least one cargo comprises a first cargo and a second cargo. In certain embodiments, the first cargo is adsorbed to the nanoparticle before the second cargo is adsorbed to the nanoparticle. In certain embodiments, the first cargo is a metal species. In certain embodiments, the metal species is Cu(II). In certain embodiments, the second cargo is a small molecule drug. In certain embodiments, the small molecule drug is rafoxanide.

In certain embodiments, the bacteria include, for example, Staphylococcus aureus/MRSA, Streptococcus pyogenes, Escherichia coli, Enterococci and/or Pseudomonas aeruginosa.

In order for the NPs to accommodate the therapeutic agents of distinct sizes and shapes, they have to be prepared under conditions that allow for tailoring the pore size/surface topography of the NPs. Therefore, in certain embodiments the dimensions of the pore are tailored by using a sol-gel method in the presence of surfactant templates.

In certain embodiments, the NP is modified at its outer surface to improve its anti-adhesion properties against biofilm-forming microorganisms.

In certain embodiments, the surfactant is a cationic surfactant. Non-limiting examples of cationic surfactants include cetyltrimethylammonium bromide (CTAB), cetyltrimethylammonium chloride (CTAC), cetylpyridinium chloride (CPC), cetylpyridinium bromide (CPB), 1-tetradecyl-3-methylimidazolium bromide (C₁₄MIMBr), 1-hexadecyl-3-methylimidazolium bromide (C₁₆MIMBr), 1-octadecyl-3-methylimidazolium bromide (C₁₈MIMBr), and 1-tetradecyloxymethyl-3-methylimidazolium chloride (C₁₄OCMIMCl), In certain embodiments, the surfactant is a non-ionic surfactant. Non-limiting examples of non-ionic surfactants include Pluronic P123, Pluronic F123, F127, Brij-76 surfactant, Triton X-100 Surfactant, and Tween 20, 40, 60 and 80 surfactants.

In certain embodiments, the surfactant is an anionic surfactant. Non-limiting examples of anionic surfactants include sodium dodecyl benzene sulfonate (SDBS), and sodium dodecyl sulphate (SDS).

In certain embodiments, the surface topography of the NP is further modified to improve its anti-adhesion properties against biofilms formed by microorganisms. Biofilm formation reduces accessibility of wounds to the therapeutic agents and hence, impeding biofilm formation can enhance the efficiency of treatment of wounds resulting in improved rate of wound healing.

In certain embodiments, the surface of NP is functionalized with at least one pH-responsive polymer and/or copolymer. Chronic wounds typically have a relatively alkaline pH environment of about 7.15 to 8.90. Depending on the pH of the skin, the NPs functionalized with the pH-responsive polymer that can swell to release the cargoes and de-swell to constrain the release of the cargoes.

In certain embodiments, the at least one pH-responsive moiety has a molecular weight less than about 1 kDa.

In certain embodiments, the NP is biocompatible.

In certain embodiments, the NP is mesoporous.

In certain embodiments, the pH-responsive moiety comprises N-(2-aminoethyl)-3-aminopropyltrimethoxysilane (DAMO).

In certain embodiments, the pH-responsive moiety is a polymer and/or copolymer. In certain embodiments, the polymer and/or copolymer is selected from the group consisting of polyethyleneimine (PEI), poly(N-isopropylacrylamide), poly(acrylic acid), poly(lactide-co-glycolide) (PLGA), polyethylene glycol, polyoxazoline, and PAMAM dendrimers.

In certain embodiments, the nanoparticle comprises a silica nanoparticle (SNP) and/or a titania nanoparticle (TNP).

In certain embodiments, the NP is a silica NP (SNP). In certain embodiments, the NP is a titania NP (TNP). In certain embodiments, the SNP is MCM-41. In certain embodiments, the SNP is MCM-48 type mesoporous silica (having a size ranging from about 8 nm to about 1,000 nm). In certain embodiments, the MCM-48 type mesoporous silica has a size selected from the group consisting of about 8, 50, 100, 150, 200, 250, 300, 350, 400, 450, 500, 550, 600, 650, 700, 750, 800, 850, 900, 950, and about 1000 nm. In certain embodiments, the SNP is SBA-15 type mesoporous silica (having a size ranging from about 8 nm to about 1,000 nm). In certain embodiments, the SBA-15 type mesoporous silica has a size selected from the group consisting of about 8, 50, 100, 150, 200, 250, 300, 350, 400, 450, 500, 550, 600, 650, 700, 750, 800, 850, 900, 950, and about 1000 nm. In certain embodiments, the SNP is large pore mesoporous silica. In certain embodiments, the SNP is colloidal silica (having a size ranging from about 8 nm to about 1,000 nm). In certain embodiments, the colloidal silica has a size selected from the group consisting of about 8, 50, 100, 150, 200, 250, 300, 350, 400, 450, 500, 550, 600, 650, 700, 750, 800, 850, 900, 950, and about 1000 nm. In certain embodiments, the SNP is surface etched colloidal silica (having a size ranging from about 8 nm to about 1,000 nm). In certain embodiments, the surface etched colloidal silica has a size selected from the group consisting of about 8, 50, 100, 150, 200, 250, 300, 350, 400, 450, 500, 550, 600, 650, 700, 750, 800, 850, 900, 950, and about 1000 nm. In certain embodiments, the SNP is KCC-1 (nanofibrous silica; having a size ranging from about 100 nm to about 1,000 nm). In certain embodiments, the KCC-1 has a size selected from the group consisting of about 100, 150, 200, 250, 300, 350, 400, 450, 500, 550, 600, 650, 700, 750, 800, 850, 900, 950, and about 1000 nm.

In certain embodiments, the TNP is mesoporous titania (having a size ranging from about 8 nm to about 1,000 nm). In certain embodiments, the mesoporous titania has a size selected from the group consisting of about 8, 50, 100, 150, 200, 250, 300, 350, 400, 450, 500, 550, 600, 650, 700, 750, 800, 850, 900, 950, and about 1000 nm. In certain embodiments, the TNP is colloidal titania (having a size ranging from about 8 nm to about 1,000 nm). In certain embodiments, the colloidal titania has a size selected from the group consisting of about 8, 50, 100, 150, 200, 250, 300, 350, 400, 450, 500, 550, 600, 650, 700, 750, 800, 850, 900, 950, and about 1000 nm.

In certain embodiments, the NP is non-porous. In certain embodiments, the NP comprises a plurality of pores. In certain embodiments, the NP is mesoporous.

In certain embodiments, the plurality of pores allow for loading of the cargo in the pores. In certain embodiments, the plurality of pores have dimensions suitable for receiving different cargoes having distinct chemical and physical (such as size and shapes) properties.

### Pharmaceutical Compositions

In another aspect, the present disclosure provides a composition. In certain embodiments, the composition comprises about 0.10% to about 0.25% of chitosan. In certain embodiments, the composition comprises about 0.10% to about 2.0% of sodium alginate. In certain embodiments, the composition comprises about 0.1% to about 0.5% of gelatin. In certain embodiments, the composition comprises about 0.01% to about 1% of grapeseed oil. In certain embodiments, the composition comprises poly-vinyl-alcohol (PVA). In certain embodiments, the composition comprises aloe vera gel. In certain embodiments, the composition comprises at least one NP of the present disclosure. In certain embodiments, the composition is formulated for application to a wound of a subject for promoting healing of the wound.

In certain embodiments, the wound is a chronic wound. In certain embodiments, the wound is a burn wound.

In certain embodiments, the composition comprises about 0.10 to about 0.25% of chitosan. In certain embodiments, the composition comprises 0.10%, 0.11%, 0.12%, 0.13%, 0.14%, 0.15%, 0.16%, 0.17%, 0.18%, 0.19%, 0.20%, 0.21%, 0.22%, 0.23%, 0.24% or 0.25% of chitosan. Chitosan is hydrophobic, known to promote tissue growth, and has bactericidal properties. Advantageously, in certain embodiments, chitosan enhances the composition's ability to heal wounds. In certain embodiments, chitosan adds mechanical stability to the film formed over the wound, since chitosan is rigid in nature.

In certain embodiments, the composition comprises about 0.10% to about 1% of sodium alginate. In certain embodiments, the composition comprises 0.10%, 0.15%, 0.20%, 0.25%, 0.30%, 0.35%, 0.40%, 0.45%, 0.50%, 0.55%, 0.60%, 0.65%, 0.75%, 0.85%, 1.0%, 1.25%, 1.5%, 1.75, or 2% of sodium alginate. Sodium alginate is a hydrophilic polymer, which is used to prevent maceration of the wound due to prolonged exposure to moisture from the wound exudate.

In certain embodiments, the composition comprises about 0.1% to about 0.5% of gelatin. In certain embodiments, the composition comprises 0.1%, 0.2%, 0.3%, 0.4%, or 0.5% of gelatin. Advantageously, in certain embodiments, gelatin promotes the proliferation of epithelial cells in skin, thereby promoting the wound healing.

In certain embodiments, the composition comprises about 0.01% to about 1% of grapeseed oil. In certain embodiments, the composition comprises 0.01%, 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, or 1.00%. Advantageously, in certain embodiments, the grapeseed oil promotes the inhibition of bacterial growth and accelerates the wound healing process especially in the inflammation phase. The acidic pH of the oil contributes to the ideal environment for fibroblastic activity, cell migration, cell proliferation, and reorganization of collagen, which aids the wound healing process.

In certain embodiments, the composition further comprises poly-vinyl alcohol (PVA). PVA is biologically inactive and has high chemical and mechanical resistance.

In other embodiments, the composition further comprises NPs of the disclosure. In yet other embodiments, the NPs of the disclosure promote healing of wounds.

In certain embodiments, the composition of the disclosure is stored inside a pressurized container as an emulsion mixture of a monomer and other components of the disclosure, wherein the emulsion is stabilized by a surfactant. In certain embodiments, the composition of the disclosure polymerizes on exposure to the air. In certain embodiments, the composition of the disclosure is delivered in the form of a foam and forms a polymeric matrix film on the skin area where it is applied to.

In certain embodiments, the polymeric matrix film is mechanically tensile and maintains its structure during movement and expansion of skin surrounding the wound.

In certain embodiments, the film remains adhered to the wound for at least about 7 days after application. In certain embodiments, the film can easily be removed if and when desired.

In certain embodiments, the composition is formulated for topical administration.

In certain embodiments, the composition is formulated for use as a cleaning agent, a disinfecting agent, a preserving agent, healing agent, a bacteriostatic agent, an antifungal agent, and/or an antiviral agent.

In certain embodiments, the composition is useful for healing of variety of wounds including but not limited to burns and chronic wounds in a subject. In certain embodiments, the composition of the disclosure can be employed in treatment of certain autoimmune diseases. In certain embodiments, the autoimmune disease includes psoriasis and eczema

Additionally, in certain embodiment, the composition of the disclosure can be incorporated in cosmetics products.

As would be understood by one of skill, an emulsion comprises a mixture of two or more immiscible liquids (i.e., contains multiple phases). Emulsions are thus distinct from solutions, which contain one or essentially only one phase. In an emulsion, one of the liquids (the dispersed phase) is dispersed in the other (the continuous phase). In one type of emulsion, a continuous liquid phase surrounds droplets of water (for example, a water-in-oil emulsion). In another type of emulsion, oil is dispersed within a continuous water phase (for example, an oil-in-water emulsion). Similarly, emulsification is the process by which emulsions are prepared.

The emulsion of the disclosure may further comprise an emulsifier. Emulsions of the disclosure may also include, but are not limited to, nanoemulsions, which are emulsions with a mean droplet size less than those of emulsions. Nanoemulsions are sometimes referred to as microemulsions and submicroemulsions. Often, the physical appearance of a nanoemulsion is transparent, rather than the often milky appearance of an emulsion, due to the reduced mean droplet size.

In one embodiment, the emulsion further comprises an emulsifier or emulgent. An emulsifier may also be a surfactant. In one embodiment, the emulsifier is a grapeseed oil. Other non-limiting examples of emulsifiers include polyglycerol polyricinoleate, sorbitan tri-stearate, polyglycerol stearate, sorbitan mono-stearate, sorbitan mono-palmitate, sorbitan mono-laurate, POE 20 sorbitan mono-laureate, POE 20 sorbitan mono-oleate, and POE 20 mono-stearate Various concentrations of an emulsifier may be used with the present disclosure. For example, the compositions of the present disclosure may comprise about 0.1%-99%, 0.1%-60%, 5%-50%, 10%-40%, 5%-25%, 10%-30%, 10%-25%, 25%-50%, 10%-75%, 25%-75%, 10%-65%, 25%-65%, 10%-60%, 25%-60%, 0.1%, 1%, 5%, 10%, 20%, 25%, 30%, 35%, 40%, 45%, 50%, 55%, 60%, 65%, 70%, 75%, 80% or any range derivable therein, of an emulsifier.

### Methods

### Wound Treatment

In another aspect, the present disclosure provides a method of healing a wound of a subject, the method comprising administering to the wound of the subject an effective amount of NP of the present disclosure and/or the composition of the present disclosure.

In certain embodiments, the wound of the subject is administered the composition of the present disclosure.

In certain embodiments, the composition is administered from a pressurized container.

In certain embodiments, the composition is stored in the container as an emulsified mixture of monomers and active ingredients stabilized by surfactants.

In certain embodiments, the wound is a chronic wound or a burn wound.

In certain embodiments, the administration prevents formation of a chronic wound.

In certain embodiments, the subject is a human.

In certain embodiments, the wound of the subject is administered the NP of the present disclosure. In certain embodiments, the NP of the present disclosure is formulated as a powder.

In certain embodiments, the wound is a chronic wound or a burn wound.

In certain embodiments, the administration prevents formation of a chronic wound.

In certain embodiments, the subject is a human.

### Preparation of a Nanoparticle (NP)

In another aspect, the present disclosure provides a method of preparing a nanoparticle of the present disclosure. In certain embodiments, the NP is functionalized with at least one pH-responsive moiety.

In certain embodiments, at least one first cargo and at least one second cargo are associated with the NP and/or the at least one pH-responsive moiety. In certain embodiments, a first cargo comprises a metal. In certain embodiments, a second cargo comprises a small molecule drug. In certain embodiments, swelling of at least one the pH-responsive moiety allows for release of the at least two cargos associated with the NP and/or at least one pH-responsive moiety.

In certain embodiments, the NP is prepared by contacting the first cargo comprising a metal with the NP functionalized with at least one pH-responsive moiety to prepare a metal-loaded NP. In certain embodiments, the NP is prepared by contacting the second cargo comprising a small molecule drug.

In certain embodiments, the first cargo comprises Cu(II).

In certain embodiments, the second cargo comprises an anthelmintic. In certain embodiments, the anthelmintic is rafoxanide.

### Kits

In one aspect, the disclosure further provides a kit comprising at least one composition of the disclosure, at least one applicator, and instructional material for use thereof. The instructional material included in the kit comprises instructions for carrying out the method of the disclosure.

In certain embodiments, disclosure provides a kit comprising a composition of the disclosure stored in a pressurized container, such that the composition of the disclosure can be directly dispensed from the container to a desired area in the form of a foam.

In another aspect, the present disclosure further provides a kit comprising the NP of the present disclosure, wherein the NP is formulated as a powder, at least one applicator, and instructional material for use thereof. The instructional material included in the kit comprises instructions for carrying out the method of the disclosure.

### Dosing

The amount of the composition of the disclosure to be administered, for example, topically, depends on the particular indication desired. For example, the dose depends on the type of wound to be treated. The dose may be different, for instance, if the delivery of the composition is intended to reduce chronic wound as opposed to burn.

The particular dosage may also be dependent on the dosing regimen chosen. For example, the composition may be delivered continuously or periodically. Conversely, the composition may be administered as a single administration as a one-time event.

The concentration and ratio of NP in the composition may vary. For example, a composition may contain a NP in a w/w ratio of from about 5 to about 95%, from about 10 to about 90%, from about 20 to about 90%, from 50 to about 90%, from about 60 to about 80%, from about 60 to about 75%, from about 20 to about 80%, from about 25 to about 75%, from about 30 to about 70%, from about 40 to about 60%, from about 1 to about 15%, from about 1 to about 10%, from about 1 to about 5%, or any range derivable therein.

Those skilled in the art recognize, or are to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

It is contemplated that any embodiment discussed in this specification can be implemented with respect to any method or composition of the disclosure, and vice versa. Furthermore, compositions of the disclosure can be used to achieve methods of the disclosure. The following examples further illustrate aspects of the present disclosure. However, they are in no way a limitation of the teachings or disclosure of the present disclosure as set forth herein.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples, therefore, specifically point out the preferred embodiments of the present disclosure, and are not to be construed as limiting in any way the remainder of the disclosure.

The experimental procedures involved to produce the materials described above are illustrated with the figures below and described with examples below.

### Materials and Methods

### Materials

Poly(ethylene glycol)-block-poly(propylene glycol)-block-poly(ethylene glycol) (or (PEG)₂₀(PPG)₇₀(PEG)₂₀ or Pluronic® 123), with an average molecular mass of ˜5800, was donated by BASF. Tetraethyl orthosilicate (TEOS), Cu(II) acetate (Cu(CH₃COO)₂), hydrochloric acid (HCl) solution (37%), and absolute ethanol (99.99%) were purchased from Fischer Scientific. N-(2-aminoethyl)-3-aminopropyltrimethoxysilane was acquired from Gelest, Inc. Rafoxanide was purchased from TCL America. Poly(ethyleneimine) solution (50% w/v) was purchased from Sigma Aldrich. All reagents were used without further purification.

### Synthesis of SBA-15 and Grafting Organic Groups on the Surface Thereof

SBA-15 was synthesized using the method described in the literature. Briefly, Pluronic® 123 (4 g) was dissolved in a solution containing HCl solution (20 mL) and distilled water (130 mL). After the temperature of the solution was adjusted to 45° C., TEOS (8.5 g) was added. The solution was vigorously stirred for 20 h, after which it was kept in an oven at 80° C. for 24 h. The solid product was recovered by filtration, washed copiously with distilled water, and dried under ambient conditions, giving as-prepared Pluronic®-containing SBA-15 mesostructured silica. Extraction of the Pluronic® 123 template from it was carried out by solvent extraction using a 1:1 solution of ethanol and diethyl ether. Briefly, 100 mg of as-synthesized SBA-15 was dispersed in 100 mL of 1:1 v/v of ethanol and diethyl ether and left stirring gently at room temperature for 5 h. Afterward, the sample was filtered and washed copiously with ethanol before leaving it to dry overnight in an oven at 60° C. This led to SBA-15-type mesoporous silica, which is labeled as SBA-15.

Organodiamine was grafted onto the surfaces of SBA-15 by stirring 100 mg of it in a solution of N-(2-aminoethyl)-3-aminopropyltrimethoxysilane (35 μL) in anhydrous toluene (60 mL) for 24 h at room temperature. The solid product was isolated via filtration and then washed with copious amounts of ethanol and dried at 50° C. overnight to provide organodiamine-functionalized SBA-15 material (i.e., DAMO/SBA).

Loading of Cu(II) Ions onto Organodiamine-Functionalized SBA-15 (Cu DAMO-SBA)

Typically, 200 mg of organodiamine-functionalized SBA-15 material (DAMO/SBA-15) was dispersed into 50 mL of 0.01 M Cu(II) acetate solution. The sample was left stirring at room temperature for 24 h and later recovered via centrifugation (7000 rpm, 15 min), and washed three times, using ethanol as a rinsing agent. The washed sample was then stored in an oven at 50° C. overnight. The resulting Cu(II) ions-loaded SBA-15 sample is labeled henceforth as Cu/DAMO-SBA.

### Loading Rafoxanide on Cu(II) Ions-Loaded SBA-15 (Cu-RFX/DAMO-SBA)

First, 200 mg of Cu/DAMO-SBA-15 was dispersed into solution of rafoxanide in toluene (30 mL, 1.6 mM or 1 mg/mL). The sample was left stirring at room temperature for 24 h and later recovered via centrifugation (7000 rpm, 15 min), and washed three times, using ethanol as a rinsing agent. The washed sample was later stored in an oven at 50° C. overnight. The resulting sample is labeled henceforth as Cu-RFX/DAMO-SBA.

### Loading Rafoxanide in Organodiamine-Modified SBA-15 (RFX/DAMO-SBA)

First, 200 mg of organodiamine-functionalized SBA-15 material (DAMO/SBA-15) was dispersed into solution of rafoxanide in toluene (30 mL, 1.6 mM or 1 mg/mL). The sample was left stirring at room temperature for 24 h and later recovered via centrifugation (7000 rpm, 15 min) and washed three times using ethanol as a rinsing agent. The washed sample was later stored in an oven at 50° C. overnight. The resulting sample is labeled henceforth as RFX/DAMO-SBA.

### Anchoring Cu(II) Ions on Rafoxanide-Loaded SBA-15 (RFX-Cu DAMO-SBA)

Typically, 200 mg of RFX DAMO-SBA-15 was dispersed into Cu(II) acetate solution (50 mL, 10 mM). The sample was left stirring at room temperature for 24 h and later recovered via centrifugation (7000 rpm, 15 min) and washed three times using ethanol as a rinsing agent. The washed sample was later stored in an oven at 50° C. overnight. The resulting sample is labeled henceforth as RFX-Cu/DAMO-SBA.

### Bactericidal Assays

The strains used in this study were Staphylococcus aureus MRSA strain USA300_LAC and Escherichia coli W3110 and their respective strains lacking copper detoxification systems, indicated herein as cop⁻ for S. aureus and LEM33 for E. coli. Strains were grown for 18 h in 2 mL of Mueller-Hinton (Sigma-Aldrich) medium in 15 mL-tubes. Broth cultures were grown at 37° C. with a shaking speed of 200 rpm. Overnight cultures were diluted to an optical density (OD₆₀₀) of 0.1, and 2.5 mL of the diluted solution was transferred to 15 mL tubes contained 20 mg of SBA-15, DAMO-SBA, Cu/DAMO-SBA, RFX/DAMO-SBA, Cu-RFX/DAMO-SBA, and RFX-Cu/DAMO-SBA nanomaterials. A control culture with no SBA-15 nanoparticles was also grown in each case, for comparison. Cells were allowed to grow for 24 h before bacteria were serially diluted in sterile phosphate-buffered saline (PBS) solution. Subsequently, 5 μL of each dilution was dropwise plated onto solid Mueller-Hinton agar (1.5%). Plates were incubated at 37° C. for 18 h before the number of viable bacteria was enumerated by counting the number of colony-forming units (CFU). Each assay was performed in triplicate.

### Release Studies of Cu(HI) Ions (Kinetic Studies)

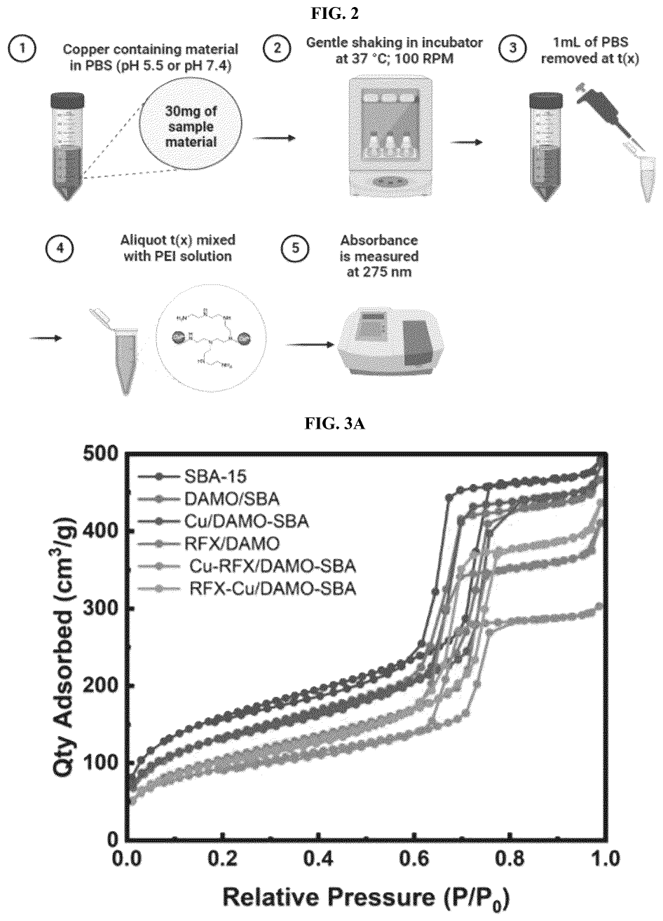

Using a literature adapted method, 30 mg of copper-containing samples were suspended in 5 mL of PBS solution (PBS 1×) for each desired time (t=0, 2, 6, 12, or 24 h) and incubated at 37° C. while spinning at 50 rpm. After each time period, 1 mL of supernatant was collected and mixed with 1 mL of polyethyleneimine (PEI) solution (1 mg/mL). All samples were individually suspended before measuring the absorbance of the formed Cu-PEI complex with a UV-Vis spectrometer at a wavelength of 275 nm. All measurements were carried out in triplicate. A summary of the process is depicted herein (FIG. 2).

### Release Studies of Cu(HI) Ions (Sustained Release)

In order to investigate the sustained release of Cu(II) ions from the nanomaterials in biological fluids, an experiment similar to that which is described elsewhere herein was performed, differing in that PBS was removed and replenished to drive the equilibrium of the system to release more Cu(II) ions. Briefly, 30 mg of Cu(II)-containing nanomaterials was placed in 5 mL of PBS 1× at pH 7.4 and 5.5. The sample was then incubated at 37° C. while spinning at 100 rpm before removing the PBS supernatant and refilling with fresh PBS. This was carried out during 5 cycles for samples where t=6 h and t=24 h. To measure the release of Cu(II) ions in each cycle, 1 mL was kept from each iteration and mixed with PEI solution (1 mg/mL), then the absorbance was measured at 275 nm after 6 h and 24 h, respectively.

### Example 1: Synthesis and Characterization

The synthesis of six exemplary mesoporous silica nanomaterials loaded with antimicrobial agents (i.e., Cu(II) ions and/or rafoxanide) is described herein. The parent material (i.e., SBA-15) is synthesized via surfactant self-assembly by following a procedure available in the literature. Solvent extraction is then used to remove the surfactant templates in it and to produce mesopores in its structures. Briefly, as-synthesized Pluronic-containing SBA-15 nanomaterials are stirred for 5 h in ethanol:diethyl ether solution at room temperature, filtered and dried. The mesopore walls of SBA-15 are then functionalized with diamino-alkyl groups using diamino-trialkoxysilane. This functional group is chosen because amine groups can easily anchor Cu(II) ions, allowing the antimicrobial agent Cu(II) to be loaded within the mesopores of SBA-15. It also enables rafoxanide to physisorb via hydrogen bonding and hydrophobic interaction.

Accordingly, the diamino-functionalized SBA-15 nanomaterial is used as a vehicle to load these two active antimicrobial agents. The structures, compositions, and bactericidal properties of the resulting nanomaterials, including Cu(II)-diamino-grafted and rafoxanide-loaded SBA-15 nanomaterials, were then characterized. Additionally, the effect of the order of addition of the active components on the antimicrobial efficacy of the nanomaterials was evaluated. Specifically, the effects of two different orders of loading were investigated (i.e., loading RFX into Cu(II)-grafted SBA-15 versus loading Cu(II) ions into RFX-loaded SBA-15).

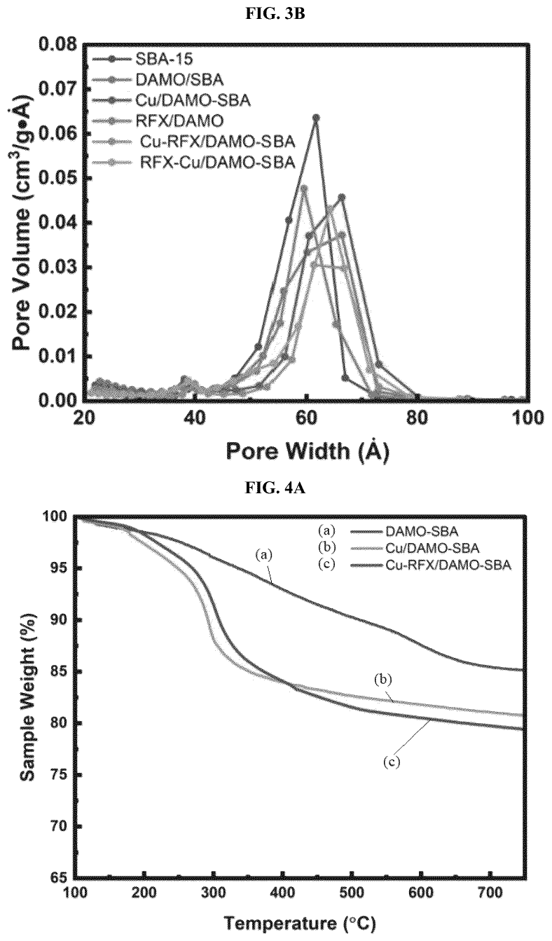

The surface areas of exemplary nanomaterials (i.e., SBA-15, DAMO/SBA, Cu/DAMO-SBA-15, RFX/DAMO-SBA, Cu-RFX/DAMO-SBA, and RFX-Cu/DAMO-SBA) were measured with N₂ porosimetry and the Brunauer-Emmett-Teller (BET) method, and their adsorption isotherms and pore size and pore volume distributions are provided herein (FIGS. 3A-3B).

The average pore sizes of the nanomaterials are 6.0, 6.1, 6.5, 6.4, 6.0, and 6.5 nm, respectively. The pore sizes barely change in most cases, especially for those in which the functional groups are on the pore walls of the nanomaterials. The surface areas of SBA-15, DAMO/SBA-15, Cu/DAMO-SBA, RFX/DAMO-SBA, Cu-RFX/DAMO-SBA, and RFX-Cu/DAMO-SBA are 656, 482, 475, 385, 327, and 370 m²/g, respectively. There is a sharp decrease in surface area from SBA-15 to DAMO/SBA which is expected, as parts of the pores in SBA-15 are occupied with diamino-alkyl ligands. This result also indirectly indicates the functionalization of the SBA-15 pore walls with these organic groups. A more subtle decrease in surface area is observed when comparing DAMO/SBA and Cu/DAMO-SBA, which suggests that Cu(II) ions are well dispersed over the inner walls of the pores, barely blocking the inner channels of the SBA-15 matrix.

The corresponding pore volumes for solvent-extracted SBA-15, DAMO/SBA Cu/DAMO-SBA-15, RFX/DAMO-SBA, Cu-RFX/DAMO-SBA, and RFX-Cu/DAMO-SBA are 0.76, 0.75, 0.77, 0.65, 0.48, and 0.69 cm³/g, respectively. The BET surface areas, pore volumes, average pore sizes, and other textural properties for the materials are provided in Table 1.

TABLE 1
Textural properties of exemplary mesoporous nanomaterials

| | Surface Area | Pore Size | Pore Volume |
|---|---|---|---|
| Materials | (m²/g) | (nm)^{a} | (cm³/g)^{a} |
| SBA-15 | 562 | 6.0 | 0.76 |
| DAMO/SBA | 482 | 6.1 | 0.75 |
| Cu/DAMO-SBA | 475 | 6.5 | 0.77 |
| RFX/DAMO-SBA | 385 | 6.4 | 0.65 |
| Cu RFX/DAMO-SBA | 327 | 6.0 | 0.48 |
| RFX-Cu/DAMO-SBA | 370 | 6.5 | 0.69 |

^{a}values are determined from desorption data

When comparing the surface areas of the DAMO/SBA and RFX/DAMO-SBA, there is a significant decrease in surface area when RFX is added into the system. This indirectly indicates that the RFX is present within the pores. This is further supported by a decrease in surface volume from 0.75 to 0.65 cm³/g. It should be noted that there is a slight increase in pore size when comparing the two, possibly because further extraction of residual surfactant template is possible when the sample is dispersed into the RFX solution with toluene. An even smaller change in surface area is observed when Cu(II) ions are added to the RFX-loaded SBA-15, suggesting that the Cu(II) ions are anchoring in the remaining exposed amine sites in the SBA-15 system. However, there is a slight increase in pore volume after this step, which is likely caused by preferential interactions between Cu(II) ions and the amine groups, pushing some RFX out of the mesopores of SBA-15.

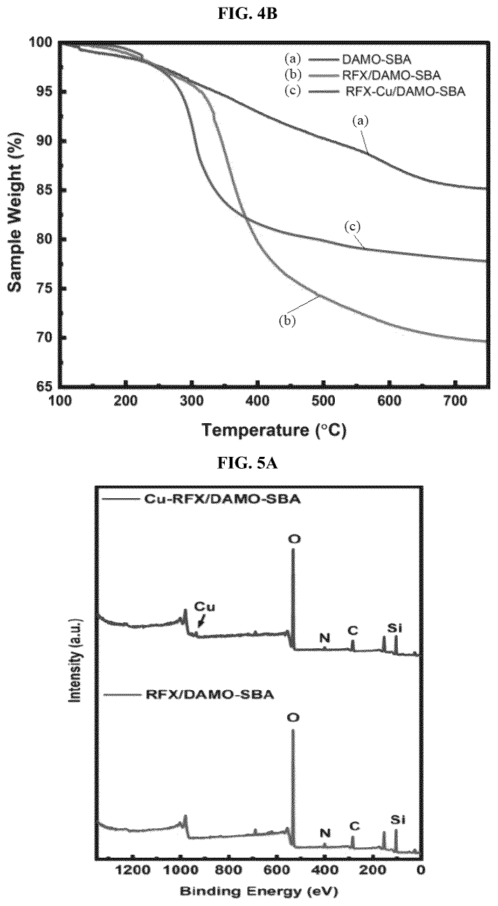

The amount of RFX loaded in DAMO/SBA is carefully determined by comparing the thermogravimetric (TG) traces of RFX/SBA and its parent material (DAMO-SBA), after normalizing the weight losses of the two curves at 100° C. (i.e., the temperature at which physisorbed water should be completely evaporated) (FIGS. 4A-4B). The comparison is done at 750° C., after most organic groups are removed from the samples. The results show that RFX/SBA has −16 wt. % more organic group than its parent material (i.e., DAMO/SBA). There are intersecting curves around 400° C. for both sets of materials (FIGS. 4A-4B) suggesting that that interaction between the organic species and Cu(II) ions/mesoporous silica may slightly change their decomposition profiles. The decomposition profiles for pure RFX and copper(II) acetate, which is the salt used to immobilize Cu(II) ions into the material. As expected, RFX completely decomposes after 500° C. as it comprises mostly organic groups. However, when it is incorporated into an inorganic carrier like SBA, the decomposition is greatly reduced, with approximately a 30% loss at 750° C.

In the case of the addition of copper species in the first step, the TG curve shows a weight loss of approximately 5%, likely due to the loss of acetate, which is the counterion of Cu(II) ion, present in the SBA matrix (FIG. 4A). This indirectly suggests that there is quite a large loading of copper(II) acetate within the SBA matrix, leaving only a small space for RFX. Without wishing to be bound by theory, this could be why only about 1% RFX can be loaded in the Cu-functionalized SBA-15 matrix. It is worth noting that the onset temperature of degradation for RFX-loaded SBA-15 shifts towards a higher temperature (330° C.) compared to that of pure RFX (200° C.). This is due to the stability imparted by the SBA-15 matrix where RFX is adsorbed into the mesoporous channel's characteristic of this material.

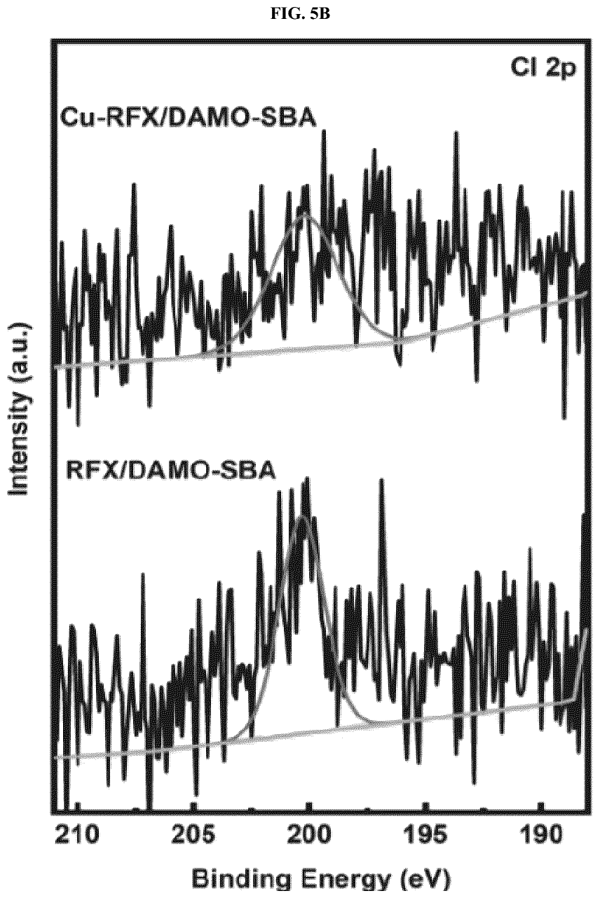

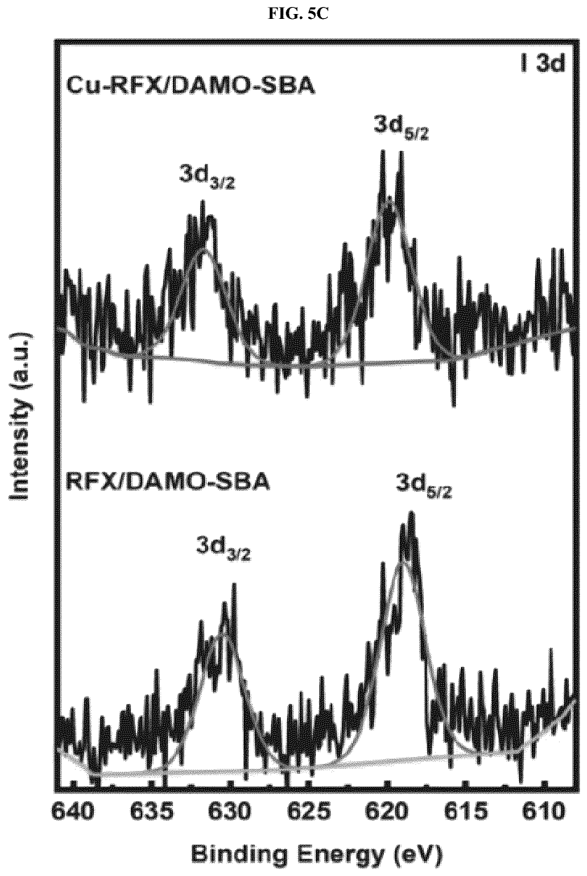

X-ray photoelectron spectroscopy (XPS) is performed to determine the chemical composition, electronic states, and nature of bonding present in the materials. High-resolution XPS scans corresponding to Cl 2p and I 3d for Cu-RFX/DAMO-SBA and RFX/DAMO-SBA are provided herein (FIGS. 5A-5C). The XPS survey spectra of Cu-RFX/DAMO-SBA and RFX/DAMO-SBA show peaks corresponding to C, N, O, Si, and I atoms. The C is spectrum of RFX/DAMO-SBA is deconvoluted to 284.45 eV, 285.69 eV, and 286.71 eV, which are attributed to C—C/C—H, C—N, and C—N⁺ species, respectively, of the diamino-alkyl groups grafted onto the SBA-15. The high-resolution XPS signals of I and Cl, which are present in rafoxanide, confirm the presence of this drug in the RFX/DAMO-SBA. The peaks at 619 eV and 630.51 eV in the I 3d XPS spectrum are respectively assigned to the 3d₅/2 and 3d₃/2 states of iodine in the C—I bond of rafoxanide. The Cl 2p spectrum has a peak at 200.32 eV which corresponds to the C—Cl bond of the rafoxanide. Additionally, the spectra of Cu-RFX/DAMO-SBA and RFX-Cu/DAMO-SBA samples are largely identical to that of RFX/DAMO-SBA except for the additional signal originating from the Cu²⁺ present in the former samples, shown in the XPS survey spectrum (FIG. 5A). The peaks at −934 eV and −954 eV in the Cu 2p XPS spectra is attributed to the 2p3/2 and 2p1/2 state of Cu²⁺ respectively.

### Example 2: Bactericidal Properties of Exemplary Nanomaterials

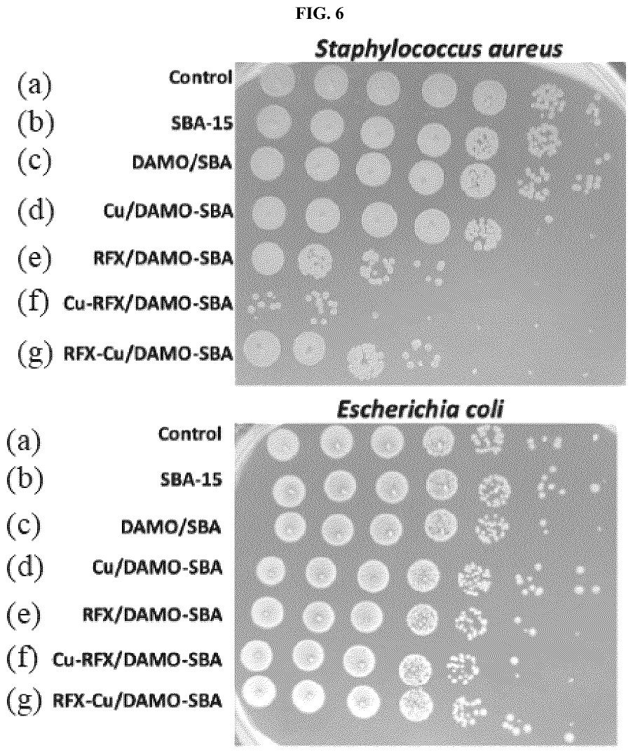

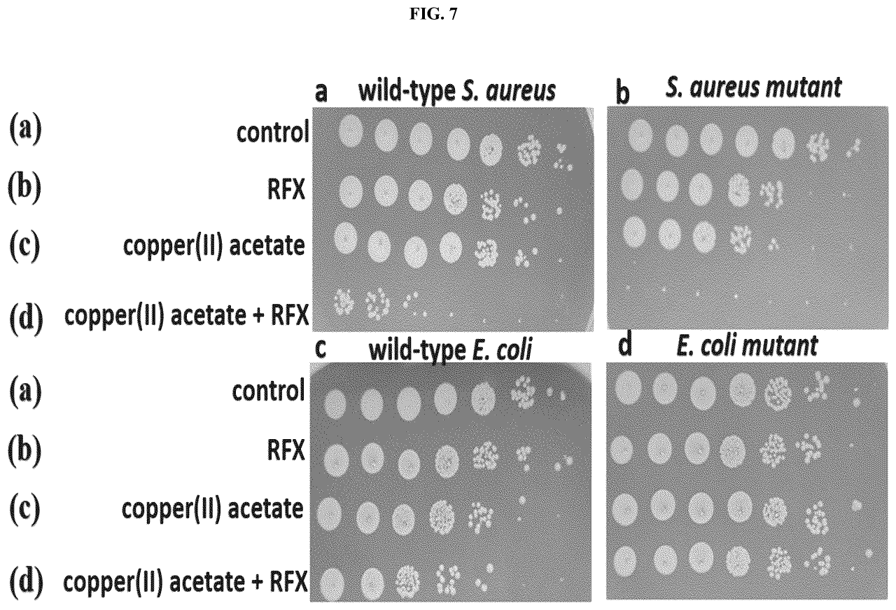

The present disclosure provides data regarding the bactericidal and/or antibacterial properties of exemplary nanomaterials against four strains of bacteria (e.g., wild-type S. aureus, copper-sensitive S. aureus, wild-type E. coli, and copper-sensitive E. coli) (FIGS. 6-7).

For both Gram-positive and Gram-negative bacteria, Cu-loaded materials do not exert any bactericidal effect on wild-type (WT) strains. Interestingly, none of the nanomaterials influenced the growth of E. coli, and there was no observed effect on this bacterium when the components of the nanomaterials are tested in their pure form. This suggests that the concentrations used in this study are not sufficient to kill E. coli.

In contrast, WT S. aureus is more susceptible to rafoxanide-loaded nanoparticles than to the same concentrations of rafoxanide. This suggests that the nanostructured SBA-15 carrier imparts has significantly different properties than the lone active component. In WT S. aureus Cu(II) species only had an additive effect on the Cu-RFX/SBA material (FIGS. 6-7). In this case, RFX is incorporated in the SBA-15 nanoparticles in the last step, after immobilizing the Cu(II) ions. When RFX/DAMO-SBA and Cu/DAMO-SBA are compared against WT S. aureus, the former has a greater antimicrobial potency indicating that RFX is the main driver behind the antimicrobial activity of the nanomaterials.

However, in presence of Cu(II) there are additive/synergistic effects increasing the antimicrobial activity of the nanomaterial (Cu-RFX/DAMO-SBA) to improve bacterial death by tenfold. This could be due to an increase in the release of RFX molecules from the nanomaterials when Cu(II) is pre-loaded in the materials. Cu-RFX/SBA nanomaterial appears to have similar effects to when the physical mixture of rafoxanide and copper are tested against WT S. aureus. On the other hand, there is almost no synergistic bactericidal effect when the Cu(II) ions are added in the last step, after RFX is loaded (i.e., RFX-Cu/DAMO-SBA). This could be due to the pores in the mesoporous being blocked by the Cu(II) species, inhibiting the release of the loaded RFX from the pores in SBA.

For cop S. aureus strains, there is no difference observed in the order of addition of Cu(II) and RFX to the nanomaterial. Notably, cells are equally responsive to both nanomaterials. This suggests that in the strain that is unable to detoxify copper it does not matter which component is released first, since the strain is unable to export copper out of the cell. Interestingly, in this organism, the physical mixture of rafoxanide and copper appears to be more potent than the same components within mesoporous nanomaterials. This suggests that the slow release of the components from the material provides an advantage to tolerate these components.

To fully determine whether the loading of RFX into the pores of SBA-15 improved the antibacterial activity of the mesoporous nanomaterials, a control experiment was performed using SBA-loaded with the same amount of RFX (as determined previously with TGA) without any other modification. Incorporating RFX into the SBA-15 matrix results in a ten-fold improvement in the RFX's antibacterial property, likely because the SBA-15 matrix can affect the RFX solubility in an aqueous solution. This is in line with some literature reports, where hydrophobic drugs have improved solubility when loaded into mesoporous materials.

To understand how the order of addition of the antimicrobial agents affects the bactericidal activity of the SBA, release studies in phosphate buffer saline (PBS), using PBS 1×, are done at different pH values under simulated biological conditions. A value of pH 5 is chosen as the other variable since methicillin-resistant S. aureus and E. coli grow via fermentation during the exponential phase and later by aerobic respiration, which primarily produces acetate. For neutral pH conditions, the release of the antimicrobial agent Cu(II) species reaches a maximum after 12 h for all three copper-containing SBA (FIGS. 6-7). Without wishing to be bound by theory, the reduction in absorbance at 24 h could be due to the system reaching equilibrium and re-adsorbing some of the Cu(II) ions from the solution.

As expected, the order of addition of copper and RFX affects the amount of the antimicrobial agent Cu(II) ions being released. Cu/SBA shows the most Cu(II) released at 12 h, followed by RFX-Cu/SBA, whose Cu(II) ions are loaded in the last step during their synthesis. The lesser release is observed for RFX-Cu/SBA, possibly because RFX impedes the release of Cu(II) ions from the nanopores of MSNs. Based on these observations, and the bactericidal results shown in FIGS. 6-7, the order of addition of the antimicrobial agents (Cu, then RFX) produces much better antimicrobial SBA even if it releases the least amount of copper among the studied nanomaterials. It should be noted that while release in PBS allows for the determination of copper ion released, it is not directly used for comparison of what is released in the bactericidal assessment, because the latter is carried out in Mueller-Hinton growth medium and contains microorganism components.

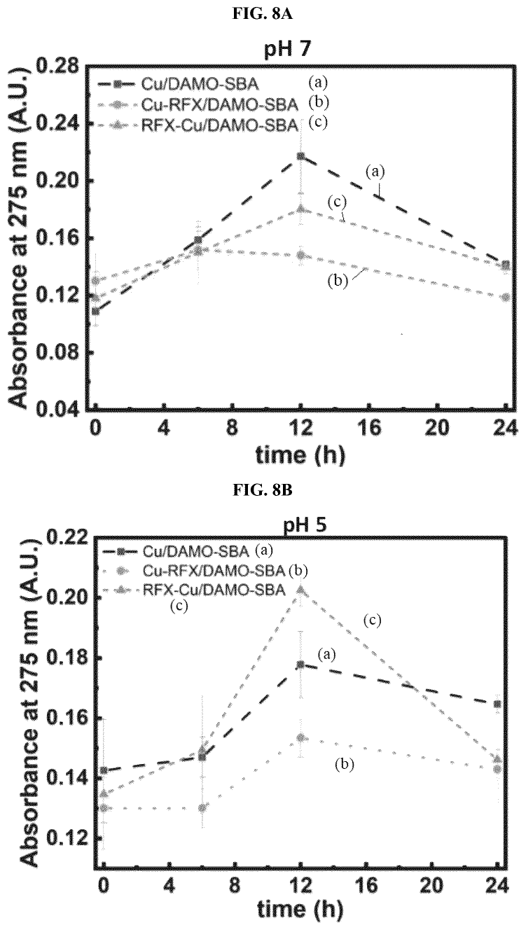

When tested at pH 5, the behavior of the nanomaterial changes slightly. Higher absorbance is shown for all samples when compared to neutral pH, but the addition of actives plays a large role in how these Cu(II) ions are released in the PBS solution. RFX-Cu/DAMO-SBA shows greater release, which could be due to competing interactions between RFX and copper with the grafted amino groups in the SBA-15 matrix (FIG. 8A). A more acidic environment also makes the grafted amino groups more cationic, thereby enabling the greater release of the positively charged Cu(II) ions (FIG. 8B). From the bactericidal assessment's perspective, the release of Cu(II) ions can be triggered when bacteria enter the last steps of their growth curves, where pH-reducing metabolites enter the media in which they are grown. It is also worth considering, that when bacteria are present, there is no possibility of reabsorption of any of the released components to the material, Cu(II) and RFX will be absorbed by the organisms. This could increase the release of the compounds from the material.

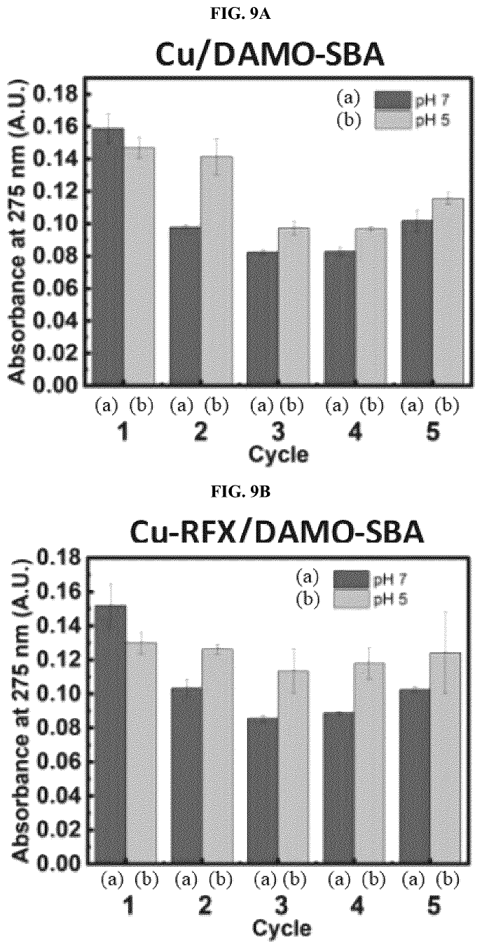

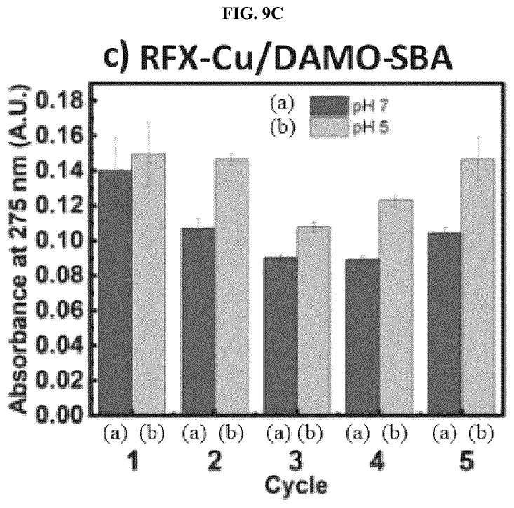

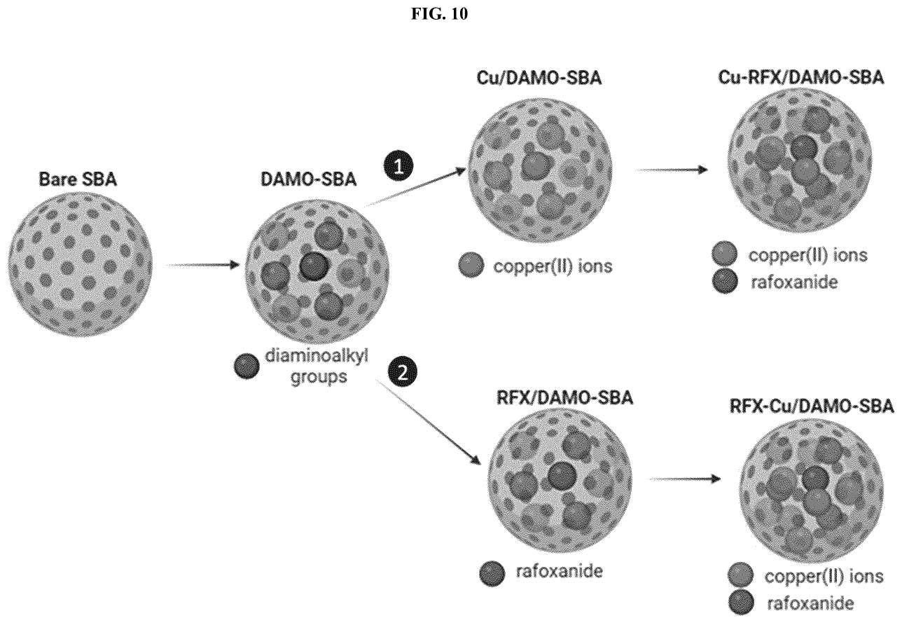

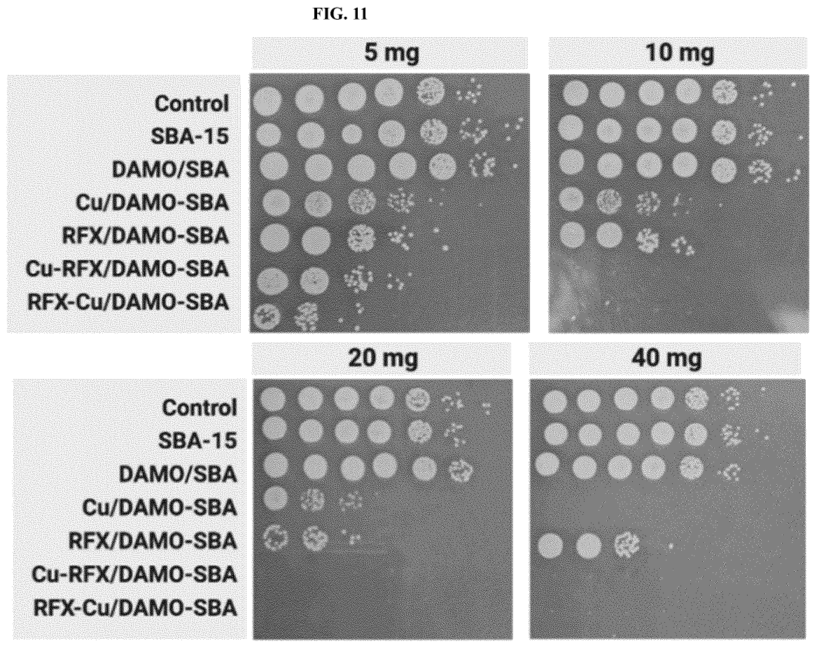

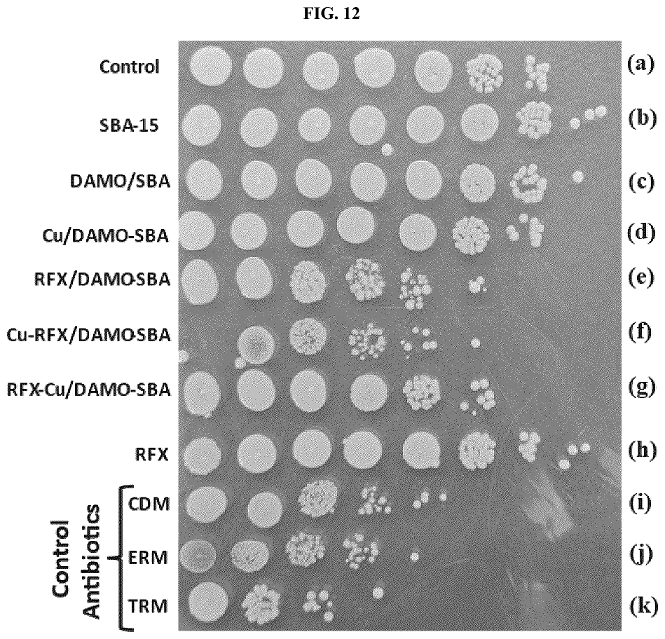

To highlight the potential benefits of using SBA-15 as a carrier for copper, the ability of nanomaterials to perform the sustained release of the two antimicrobial agents is examined over 5 cycles. Briefly, 30 mg Cu(II)-containing samples are incubated at the 37° C. and 100 rpm for 6 h in PBS 1× before replacing the solution with fresh PBS at pH 7.4 and pH 5.5. This was repeated over 5, 6 h cycles. Steep declines in Cu(II) ion release between the first and second cycles at pH 7, with smaller changes in absorbance between cycles 2 through 4 (FIGS. 9A-9C). At pH 5, there are less drastic changes between cycles, suggesting that there is a more sustained release under acidic conditions throughout 5 cycles. Interestingly, Cu-RFX/DAMO-SBA, the best-performing material, has a more sustained release, as shown by the small decreases in absorbance at pH 5 across all cycles, as shown in FIG. 9B.

Running the materials through different cycles in conditions that would simulate where equilibrium is not reached (i.e., where bacteria interact with the materials) is useful for understanding the release in the presence of bacteria, which transport Cu(II) ions through their membranes. However, it should be noted that more research is necessary, perhaps in growth culture, to ascertain whether the behavior is the same.

### ENUMERATED EMBODIMENTS

The following exemplary embodiments are provided, the numbering of which is not to be construed as designating levels of importance:

Embodiment 1 provides a nanoparticle (NP) functionalized with at least one pH-responsive moiety, wherein:
(a) at least two cargos are associated with the NP and/or the at least one pH-responsive moiety,
   wherein each cargo is independently selected from the group consisting of a therapeutic cargo, a molecular marker, and a biomarker; and
(b) swelling of at least one the pH-responsive moiety allows for release of the at least two cargos associated with the NP and/or at least one pH-responsive moiety.

Embodiment 2 provides the NP of Embodiment 1, wherein the therapeutic cargo is at least one selected from the group consisting of a small molecule drug, a metal species, and a therapeutic peptide,
optionally wherein the therapeutic cargo comprises at least one selected from the group consisting of a cleaning agent, disinfecting agent, preserving agent, healing agent, bacteriostatic agent, antifungal agent, and antiviral agent.

Embodiment 3 provides the NP of Embodiment 2, wherein the small molecule drug is a GroEL/ES inhibitor.

Embodiment 4 provides the NP of Embodiment 3, wherein the GroEL/ES inhibitor is selected from the group consisting of hydroxybiphenylamide and sulfamido-2-arylbenzoxazole, or an analogue thereof, and any combination thereof.

Embodiment 5 provides the NP of Embodiment 2, wherein the small molecule drug is an anthelmintic.

Embodiment 6 provides the NP of Embodiment 5, wherein the anthelmintic is rafoxanide.

Embodiment 7 provides the NP of any one of Embodiments 2-6, wherein the metal species is Cu(II).

Embodiment 8 provides the NP of any one of Embodiments 1-2 and 5-7, wherein the cargo comprises rafoxanide and Cu(II).

Embodiment 9 provides the NP of Embodiment 8, wherein the NP is prepared by loading the Cu(II) into the NP before loading the rafoxanide into the NP.

Embodiment 10 provides the NP of any one of Embodiments 1-9, wherein the molecular marker and/or biomarker is selected from the group consisting of O₂, β-catenin, c-myc, and matrix metalloproteinases.

Embodiment 11 provides the NP of any one of Embodiments 1-10, wherein at least one of the following applies:
(a) the NP has a plurality of pores having dimensions which are controlled by preparing a silica matrix by a sol-gel method in the presence of a surfactant template;
(b) the NP is modified at its outer surface to improve its anti-adhesion properties against biofilm-forming microorganisms;
(c) the at least one pH-responsive moiety has a molecular weight less than 1 kDa;
(d) the NP is biocompatible; and
(e) the NP is mesoporous.

Embodiment 12 provides the NP of any one of Embodiments 1-11, wherein the pH-responsive moiety is N-(2-aminoethyl)-3-aminopropyltrimethoxysilane (DAMO).

Embodiment 13 provides the NP of any one of Embodiments 1-11, wherein the pH-responsive moiety is a polymer and/or copolymer.

Embodiment 14 provides the NP of Embodiment 13, wherein the polymer and/or copolymer is selected from the group consisting of polyethyleneimine (PEI), poly(N-isopropylacrylamide), poly(acrylic acid), poly(lactide-co-glycolide) (PLGA), polyethylene glycol, polyoxazoline, and PAMAM dendrimers.

Embodiment 15 provides the NP of any one of Embodiments 1-14, wherein the nanoparticle comprises a silica nanoparticle (SNP) or a titania nanoparticle (TNP).

Embodiment 16 provides the NP of any one of Embodiments 1-15, wherein the nanoparticle comprises one of the following:
(a) a porous nanoparticle comprising a plurality of pores, which allow for loading of the cargos in the plurality of pores; or
(b) a non-porous nanoparticle.

Embodiment 17 provides the NP of Embodiment 15 or 16, wherein one of the following applies:
(a) the SNP is selected from the group consisting of MCM-41, MCM-48 type mesoporous silica (having size ranging from about 8 nm to about 1,000 nm), SBA-15 type mesoporous silica (having size ranging from about 8 nm to about 1,000 nm), large pore mesoporous silica, colloidal silica (having size ranging from about 8 nm to about 1,000 nm), surface etched colloidal silica (having size ranging from about 8 nm to about 1,000 nm), and KCC-1 (nanofibrous silica having size ranging from about 100 nm to about 1,000 nm); or
(b) the TNP is selected from the group consisting of mesoporous titania (having size ranging from about 8 nm to about 1,000 nm), and colloidal titania (having size ranging from about 8 nm to about 1,000 nm).

Embodiment 18 provides a composition comprising:
(a) about 0.10% to about 0.25% of chitosan;
(b) about 0.10% to about 2.0% of sodium alginate;
(c) about 0.1% to about 0.5% of gelatin;
(d) about 0.01% to about 1% of grapeseed oil;
(e) poly-vinyl-alcohol (PVA);
(f) aloe vera gel; and
(g) at least one NP of any one of Embodiments 1-17;
   wherein the composition is formulated for application to a wound of a subject for promoting healing of the wound.

Embodiment 19 provides the composition of Embodiment 18, wherein the wound comprises a burn wound or a chronic wound.

Embodiment 20 provides the composition of Embodiment 18 or 19, wherein upon application of the composition to the wound the composition forms a thin film on the wound.

Embodiment 21 provides the composition of Embodiment 20, wherein the film is mechanically tensile and maintains its structure during movement and expansion of skin surrounding the wound.

Embodiment 22 provides the composition of Embodiment 20 or 21, wherein the film can remain adhered to the wound for at least about 7 days after application.

Embodiment 23 provides the composition of any one of Embodiments 18-22, wherein the composition is applied to the wound as a foam.

Embodiment 24 provides the composition of any one of Embodiments 18-23, wherein the composition is formulated for topical administration.

Embodiment 25 provides the composition of any one of Embodiments 18-24, wherein the composition is formulated for use as a cleaning agent, disinfecting agent, preserving agent, healing agent, bacteriostatic agent, antifungal agent, and/or antiviral agent.

Embodiment 26 provides a method of healing a wound of a subject, the method comprising administering to the wound of the subject an effective amount of the NP of any one of Embodiments 1-17 and/or the composition of any one of Embodiments 18-25.

Embodiment 27 provides the method of Embodiment 26, wherein the wound of the subject is administered the composition of any one of Embodiments 18-25.

Embodiment 28 provides the method of Embodiment 27, wherein the composition is administered from a pressurized container.

Embodiment 29 provides the method of Embodiment 27 or 28, wherein the composition is stored in the container as an emulsified mixture of monomers and active ingredients stabilized by surfactants.

Embodiment 30 provides the method of Embodiment 26, wherein the wound of the subject is administered the NP of any one of Embodiments 1-17.

Embodiment 31 provides the method of Embodiment 30, wherein the NP is formulated as a powder.

Embodiment 32 provides the method of any one of Embodiments 26-31, wherein the wound is a chronic wound or a burn wound.

Embodiment 33 provides the method of any one of Embodiments 26-31, wherein the administration prevents formation of a chronic wound.

Embodiment 34 provides the method of any one of Embodiments 26-33, wherein the subject is a human.

Embodiment 35 provides a kit for healing wounds and/or preventing chronic wounds, the kit comprising the composition of any one of Embodiments 18-25 stored in a pressurized container and instructional material for use thereof.

Embodiment 36 provides a kit for healing wounds and/or preventing chronic wounds, the kit comprising the NP of any one of Embodiments 1-17, wherein the NP is formulated as a powder.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A nanoparticle (NP) functionalized with at least one pH-responsive moiety, wherein:
(a) at least two cargos are associated with the NP and/or the at least one pH-responsive moiety,
wherein each cargo is independently selected from the group consisting of a therapeutic cargo, a molecular marker, and a biomarker; and
(b) swelling of at least one the pH-responsive moiety allows for release of the at least two cargos associated with the NP and/or at least one pH-responsive moiety.

2. The NP of claim 1, wherein the therapeutic cargo is at least one selected from the group consisting of a small molecule drug, a metal species, and a therapeutic peptide,
optionally wherein the therapeutic cargo comprises at least one selected from the group consisting of a cleaning agent, disinfecting agent, preserving agent, healing agent, bacteriostatic agent, antifungal agent, and antiviral agent.

3. The NP of claim 2, wherein the small molecule drug is a GroEL/ES inhibitor or an anthelmintic drug.

4. The NP of claim 3, wherein one of the following applies:
(a) the GroEL/ES inhibitor is selected from the group consisting of hydroxybiphenylamide and sulfamido-2-arylbenzoxazole, or an analogue thereof, and any combination thereof, or
(b) the anthelmintic drug is rafoxanide.

5. The NP of claim 2, wherein the metal species is Cu(II).

6. The NP of claim 1, wherein the cargo comprises rafoxanide and Cu(II).

7. The NP of claim 6, wherein the NP is prepared by loading the Cu(II) into the NP before loading the rafoxanide into the NP.

8. The NP of claim 1, wherein the molecular marker or biomarker is selected from the group consisting of O₂, β-catenin, c-myc, and matrix metalloproteinases.

9. The NP of claim 1, wherein at least one of the following applies:
(a) the NP has a plurality of pores having dimensions which are controlled by preparing a silica matrix by a sol-gel method in the presence of a surfactant template;
(b) the NP is modified at its outer surface to improve its anti-adhesion properties against biofilm-forming microorganisms;
(c) the at least one pH-responsive moiety has a molecular weight less than 1 kDa;
(d) the NP is biocompatible;
(e) the NP is mesoporous; and
(f) the molecular marker and/or biomarker is selected from the group consisting of O₂, β-catenin, c-myc, and matrix metalloproteinases.

10. The NP of claim 1, wherein at least one of the following applies:
(a) the pH-responsive moiety is N-(2-aminoethyl)-3-aminopropyltrimethoxysilane (DAMO); and
(b) the pH-responsive moiety is a polymer or copolymer, optionally wherein the polymer or copolymer is selected from the group consisting of polyethyleneimine (PEI), poly(N-isopropylacrylamide), poly(acrylic acid), poly(lactide-co-glycolide) (PLGA), polyethylene glycol, polyoxazoline, and PAMAM dendrimers.

11. The NP of claim 1, wherein the nanoparticle comprises a silica nanoparticle (SNP) or a titania nanoparticle (TNP).

12. The NP of claim 11, wherein one of the following applies:
(a) the SNP is selected from the group consisting of MCM-41, MCM-48 type mesoporous silica (having size ranging from about 8 nm to about 1,000 nm), SBA-15 type mesoporous silica (having size ranging from about 8 nm to about 1,000 nm), large pore mesoporous silica, colloidal silica (having size ranging from about 8 nm to about 1,000 nm), surface etched colloidal silica (having size ranging from about 8 nm to about 1,000 nm), and KCC-1 (nanofibrous silica having size ranging from about 100 nm to about 1,000 nm); or
(b) the TNP is selected from the group consisting of mesoporous titania (having size ranging from about 8 nm to about 1,000 nm), and colloidal titania (having size ranging from about 8 nm to about 1,000 nm).

13. A composition comprising:
(a) about 0.10% to about 0.25% of chitosan;
(b) about 0.10% to about 2.0% of sodium alginate;
(c) about 0.1% to about 0.5% of gelatin;
(d) about 0.01% to about 1% of grapeseed oil;
(e) poly-vinyl-alcohol (PVA);
(f) aloe vera gel; and
(g) at least one nanoparticle (NP) of claim 1;
wherein the composition is formulated for application to a wound of a subject for promoting healing of the wound.

14. The composition of claim 13, wherein the wound comprises a burn wound or a chronic wound, optionally wherein upon application of the composition to the wound the composition forms a thin film on the wound, and optionally wherein at least one of the following applies:
(a) the film is mechanically tensile and maintains it structure during movement and expansion of the skin surrounding the wound;
(b) the film is mechanically tensile and maintains its structure during movement and expansion of skin surrounding the wound; and
(c) the film can remain adhered to the wound for at least about 7 days after application.

15. The composition of claim 13, wherein at least one of the following applies:
(a) the composition is applied to the wound as a foam;
(b) the composition is formulated for topical administration; and
(c) the composition is formulated for use as a cleaning agent, disinfecting agent, preserving agent, healing agent, bacteriostatic agent, antifungal agent, and/or antiviral agent.

16. A method of healing a wound of a subject, the method comprising administering to the wound of the subject an effective amount of the nanoparticle (NP) of claim 1.

17. A method of healing a wound of a subject, the method comprising administering to the wound of the subject an effective amount of the composition of claim 13, optionally wherein the composition is administered from a pressurized can, optionally wherein the composition is stored in the container as an emulsified mixture of monomers and active ingredients stabilized by surfactants.

18. The method of claim 16, wherein at least one of the following applies:
(a) the NP is formulated as a powder;
(b) the wound is a chronic wound or a burn wound;
(c) the administration prevents formation of a chronic wound; and
(d) the subject is a human.

19. A kit for healing wounds and/or preventing chronic wounds, the kit comprising the composition of claim 13 stored in a pressurized container and instructional material for use thereof.

20. A kit for healing wounds or preventing chronic wounds, the kit comprising the NP of claim 1, wherein the NP is formulated as a powder.
